(19) United States
(12) Patent Application Publication
(10) Pub. No.: US 2002/0107503 A1
(43) Pub. Date: Aug. 8, 2002

(54) Intermittent drug delivery method and system

(76) Inventor: Lucas S. Gordon, Sammamish, WA (US)

Correspondence Address: LAW OFFICES OF RONALD M ANDERSON, 600 108TH AVE, NE, SUITE 507, BELLEVUE, WA, 98004, US

(21) Appl. No.: 09/778,222

(22) Filed: Feb. 6, 2001

(51) Int. Cl. A61M 31/00

(52) U.S. Cl. 604/507; 606/202

(57) ABSTRACT

A method and system for delivering a medicinal agent, such as a therapeutic drug or diagnostic agent, to a treatment site within a limb of a patient. An infusion catheter is inserted into a blood vessel and advanced to the treatment site. To prevent blood flow through the treatment site from carrying away the medicinal agent, the blood flow in the limb is stopped by applying external pressure with a constriction device, such as a pressure cuff coupled to an inflation pump, or a tourniquet. The medicinal agent is then injected through the catheter with an infusion pump while the blood flow is stopped. Optionally, the infusion process is repeated in successive cycles that are separated by a rest period in which blood flow in the limb is allowed to resume. A controller can be used to automate the process.

### FIELD OF THE INVENTION

The present invention is generally directed to a medical method and system to deliver medicinal agents in veins and arteries, and more specifically, is directed to intermittent localized retrograde delivery of medicinal agents in veins and capillaries to provide reduced concentrations of medicinal agents within local areas of a patient's body.

### BACKGROUND OF THE INVENTION

Treatment of diseases that affect peripheral areas of a human body is often a difficult task. Advances in cardiology have involved a retrograde (i.e., opposite the direction of blood flow) delivery of medications into areas of the body affected by poor arterial circulation. One such advance is retrograde perfusion, a method of delivering, in the retrograde direction, drugs, solutions, or blood to a tissue area. During cardiopulmonary bypass, retrograde perfusion is used to deliver cardioplegic solutions into cardiac veins and tissue. Corday et al., in U.S. Pat. No. 4,689,041, entitled, “Retrograde Delivery of Pharmacologic and Diagnostic Agents Via Venous Circulation” (hereinafter “Corday”), describe a method using a catheter having a balloon disposed on its distal end, for retrograde venous injection of various fluids into a blockaded region made inaccessible by an occluded artery. While aiding in the retrograde delivery of fluids into the veins, venules and capillaries of the heart, Corday does not provide a method for successful retrograde delivery of fluid in other venous systems. Unlike the heart, most other areas of the body have veins that are interconnected to form an outflow path or grid with multiple, parallel, interconnecting vessels. If retrograde perfusion is attempted in these areas using the Corday technique, the infused fluid merely flows into a parallel vein and away from the capillary vessels, so that retrograde flow of the fluid into the target capillary system does not occur. The capillaries are the optimal blood vessels for drug delivery due to their ultra-thin walls, providing maximum delivery of the drug into the surrounding tissue.

Diabetes, a disease that causes restricted blood flow and arterio-venous shunting in peripheral limbs, leads to infections and ulcers that are slow to heal. Antibiotics applied to the exterior surface of the ulcers have been relatively ineffective due to an inability of the medication to penetrate deeply into tissue surrounding infected areas. An alternative method for treating these infections is a systemic administration of antibiotic agents into the venous system of the infected limb. Unfortunately, concentrations of antibiotics at a level appropriate to treat the infection often cause toxic effects in other areas of the body.

Diabetic foot ulcers have been effectively treated with a regional administration of high concentrations of antibiotics. Cavini-Ferreira et al. report the use of venous infusion of an antibiotic for infected, diabetic foot ulcers (Cavini-Ferreira, P.C., “Retrograde Venous Perfusion in the Diabetic Foot,” Ischemic Diseases and the Microcirculation (1989), K. Messmer. Munchen, pages 92-99). In part, the method of Cavini-Ferreira uses a technique for circulatory arrest as described by Bier in “Ueber einen neuen Weg Localanasthesie and den Gliedmaassen zu erzeugen,” Archiv klinishe Chirugie 86 (1908), pages 1007-1016. Bier proposed a method of performing local anesthesia to a limb by applying a tourniquet inflated to a pressure above that of the arterial blood flow. The result is complete stasis of the circulatory system in the limb into which an anesthetic agent is injected, resulting in anesthesia in the limb. Cavini-Ferreira et al. used Bier's circulatory arrest technique in conjunction with injection of an antibiotic medication mixed with a large volume of liquid through a needle inserted into a superficial vein on the dorsum of the foot. This treatment was repeated once very 24 hours, for 2 to 11 days. Each treatment required a new cannulation of the vein for antibiotic administration. The voluminous injection results in expansion and flooding of the venous system within the foot and leg. The circulatory arrest was maintained for periods of 20 minutes. When blood flow to an area is reduced or stopped, oxygen deprivation or hypoxia occurs. It is generally believed that ischemia or loss of blood flow to tissue, excluding the heart and brain, may be maintained safely for a period of up to 30 minutes. However, diabetic patients suffer from circulatory abnormalities that include increased arterial-venous shunting in the feet, resulting in lower blood flow to the tissues and hypoxia. This reduction of blood flow is a primary reason that ulcers develop in these locations. It is reasonable to assume that the safe ischemic period for the feet of diabetic patients is much less than 30 minutes and should be minimized.

Blood stasis for long lengths of time may also place the patient at increased risk of thrombosis, although the use of systemic heparinization may lengthen the safe period of arrest. Although the extended arrest technique of Cavini-Ferreira et al. was effective in healing infected diabetic foot ulcers, many patients reported at least moderate pain during the procedure. Cavini-Ferreira et al. do not describe or suggest how this technique could be used for more localized or intermittent drug delivery into the arteries, veins, and capillaries of the infected tissue.

U.S. Pat. No. 5,254,087 (McEwan) entitled, “Tourniquet Apparatus For Intravenous Regional Anesthesia” (hereinafter referred to as “McEwan”) describes apparatus designed for administering and maintaining anesthesia (Bier's circulatory arrest) in a portion of a patient's limb distal to a cuff using a cuff pressure, transducers for generating a pressure signal representative of the maximum pressure applied to the vein by the cuff, delivery pressure control means responsive to the applied pressure signal for determining a reference pressure and for generating a delivery pressure control signal representative of the reference pressure, and anesthetic delivery means. This system attempts to insure that anesthetic is maintained within the limb during surgery. The anesthetic is delivered into a superficial vein using a cannula. However, McEwan anesthetizes a whole limb, or a region of the limb in which blood flow may unnecessarily be halted. No attempt is made to localize the anesthetic to a particular defined location within the limb. In fact, methods are described for removing as much blood from the limb as possible in order to introduce a maximal amount of anesthetic agent into the entire limb. This raises the risks and discomfort involved with denying blood to the limb tissues. Also, McEwan does not describe or suggest administration of any therapeutic or diagnostic agents into the limb, but instead, only describes administration of an anesthetic agent. The long occlusion times required for surgery are acknowledged to be painful to the patient, and methods using dual-bladder cuffs are described to reduce this pain. However, the McEwan does not discuss intermittent delivery to prevent or reduce this pain or to reduce the effects of ischemia. This fact is not surprising, since the basis of the McEwan patent is to prevent any escape of anesthetic into general circulation. Generally, this surgical procedure is intended to be performed only once, therefore, McEwan makes no attempt to develop methods or equipment suitable for multiple cannulations of the anesthetic administration site. In any case, it is not desirable to require multiple cannulations, because of increased infection risks and discomfort to the patient.

Patents to Calderon, including U.S. Pat. Nos. 4,883,459; 4,867,742; and 4,714,460, disclose various schemes for the retrograde profusion of a tumor using a catheter system that includes a suction lumen and an infusion lumen. Seals are associated with each lumen. The infusion seal includes a balloon that is disposed between an outlet port of the infusion lumen and a port of the suction lumen for use in sealing a patient's vein. Similarly, the suction seal comprises a balloon disposed on the catheter proximal the port of the suction lumen, for preventing fluid flow through the vein.

In U.S. Pat. No. 4,883,459, Calderon teaches that a carrier medium is injected through the infusion lumen into the vein at a desired flow rate and pressure until a steady-state flow is established. Next, a second, less dense carrier medium is injected through the infusion lumen. The back pressure on the carrier medium is increased when the second carrier medium is at the tumor, forcing the second carrier medium into interstitial spaces in the tumor at an attack site. Next, an active ingredient is injected behind a carrier fluid into the patient's vein from the infusion lumen along the established flow path, and a back pressure on a carrier fluid and active ingredient is increased when the active ingredient is at the attack site, forcing the active ingredient into the interstitial spaces within the tumor. The active ingredient is then collected through the suction lumen after its profusion through the tumor, preventing the active ingredient, which is a chemotherapy drug of potential toxicity to the remainder of the patient's body, from being circulated throughout the patient's circulatory system. The other two Calderon patents claim various related aspects of this basic concept. This concept helps to localize the treatment. However, the seals may be difficult to establish and maintain accurately, which may allow the injected fluid to leak around the seals. Also, because the blood flow is only stopped in the small area between seals, nearby blood flow may allow the injected fluid to leak away from the tumor through nearby return veins, or beyond the interstitial spaces within the tumor. The suction lumen may also unnecessarily extract blood from nearby vessels rather than just the injected fluid.

U.S. Pat. No. 5,069,662 (Bodden); U.S. Pat. No. 5,411,479 (Bodden); and U.S. Pat. No. 5,817,046 (Glickman) disclose several inventions pertaining to apparatus for isolating fluid flow into and out of the body of a patient, to enable a high concentration of a chemotherapeutic agent to be profused for treating a tumor within the pelvic region of a patient. The two Bodden patents disclose a catheter having spaced-apart balloon sections that can be inflated in a blood vessel to isolate a body organ that contains a tumor. The catheter includes a lumen for withdrawing blood from the organ into which a high concentration of an agent used to treat the cancerous tumor has been injected. Blood is thus extracted from the organ through an isolated section of the vascular system that is coupled to the organ's blood supply and is circulated through a filter to remove the toxic anti-cancer agent before being returned to the patient's body. This process prevents toxic levels of the anti-cancer drug from entering the general circulatory system of the patient. However, because this method primarily intends a high dose of a toxic agent, such as a chemotherapy drug, this method requires the blood to be removed, filtered, and re-infused. Doing so is considerably complex and not suitable for small, intermittent doses that do not have such adverse effects in general circulation as the high doses intended by Bodden.

A related system is disclosed by Glickman for treating a tumor in the pelvic cavity. In the Glickman invention, bilateral thigh tourniquets are applied to interrupt blood flow into the legs of the patient. Furthermore, balloon catheters are inserted into the patient's body and positioned and inflated to occlude blood flow through the aorta and the vena cava at a point above the pelvic region. Then, additional catheters are inserted to enable blood to be withdrawn from the pelvic cavity and circulated through a filter that removes a chemotherapeutic agent from the blood. Although Glickman mentions the possibility of adapting the apparatus disclosed in his patent to treatment of tumors within other portions of the body, there is no clear explanation of how this can be accomplished. It also appears that use of Glickman's apparatus for treating tumors in the leg would require a balloon catheter to occlude blood flow above the tumor and a tourniquet to occlude blood flow below the tumor, which would be of little use in treating a tumor disposed in a foot or other location that could not readily be isolated between a tourniquet and a balloon catheter.

From the preceding description of various approaches developed in the prior art for isolating and administering treatment to a specific site in a patient's body, it will be evident that there remains a need for a safe and effective method and system that permits the localized and repeated delivery of therapeutic or diagnostic agents into a site and which takes advantage of retrograde perfusion, but avoids the problems occurring due to capillary shunting. In particular, there is a need for a method and system that will enable the localized delivery of a medicinal fluid directly at the site of an infection in capillaries of limbs without causing severe pain to the patient and with little risk of clot formation.

### SUMMARY OF THE INVENTION

The present invention provides a method for delivering a medicinal agent, such as a therapeutic drug or diagnostic agent, to a treatment site within a patient's limb. The method includes the steps of inserting an infusion catheter into the patient's vascular system, either within a vein or an artery, and advancing the catheter to the treatment site. Placement of the catheter in a vein rather than an artery is usually preferred because of easier visualization and access, minimal atherosclerotic plaque buildup, and easier control of bleeding after catheter removal. However, the catheter can also be placed within an artery, if desired. The distal tip of the catheter is advanced as close as possible to capillaries disposed in the treatment site. Thus, in a vein, the distal tip of the catheter is advanced in a retrograde direction relative to normal blood flow, and in an artery, the distal tip is advanced in an antegrade direction relative to the normal direction of blood flow. To prevent loss of the medicinal agent from the treatment site due to blood flow, the blood flow into and out of the treatment site within the limb is stopped by applying external pressure with a constriction device placed on the limb proximal of the treatment site. Preferably, the constriction device is a pressure cuff, tourniquet, or similar device. (Note that as used herein in regard to a patient's limb, the term “proximal” refers to a location that is closer to the torso of the patient's body than the treatment site, while the term “distal” as applied to a limb refers to a location that is closer to the tip of the limb.) After the flow of blood has been occluded by the constriction device, a small quantity of the medicinal agent is injected through the catheter. Since the catheter tip is disposed proximate the treatment site, only a relatively small amount of the medicinal agent need be locally infused into the capillaries to provide the desired levels of the medicinal agent at the treatment site.

Occluding the blood flow into and out of the treatment site of the limb also enables the medicinal agent to perfuse into target tissue of the treatment site more effectively than would a medicinal agent injected in flowing blood, or at a location remote from the treatment site. After a predetermined perfusion period has lapsed, the constriction device is released, allowing normal blood flow through the vascular system to resume, to re-oxygenate the limb. The occlusion time is limited to the time during which blood flow into and out of the limb can safely be interrupted and to the time necessary to control pain felt by the patient during the occlusion. Excessive stagnation of blood within the limb may lead to possible thrombosis, although the occlusion time may be extended with the use of standard anticoagulants such as heparin. The infusion process is repeated as often as desired to provide a desired perfusion of the medicinal agent into the surrounding tissue at the treatment site. Since each dose of the medicinal agent is very small, the systemic buildup of the medicinal agent is minimal, even after several doses have been administered. A typical sequence would be four minutes during which blood flow is occluded and the medicinal agent is infused, followed by releasing the flow restriction for one minute to enable blood to again flow into and out of the limb. The repetitive sequence of occluding the flow of blood, injecting the agent, waiting a specified period of time, and re-establishing the blood flow for a specified period of time may be done manually. However, automatically performing these steps is simpler and more reliable.

Another aspect of the invention is a system for delivering a medicinal agent to a treatment site within a patient. The system includes an infusion catheter and an external constrictor. The system may also preferably include a delivery device for infusing the medicinal agent. An introducer sheath is preferably included and can be inserted into the patient's vein or artery to provide easier and reusable access for inserting the catheter. The catheter is suitable for placement within a vein or artery, and includes at least one infusion lumen extending from an external proximal port to an internal distal port. The catheter may include a radio opaque element disposed adjacent to its distal end to assist in routing the catheter through the vascular system and positioning the distal end of the catheter at the treatment site. To further assist in routing and positioning, the catheter may also include a second lumen adapted to receive a guide wire. The catheter may also optionally further include an enlarged portion adjacent to its distal tip that is adapted to wedge against the inside wall of the vessel to prevent the medicinal agent from flowing away from the treatment site, past the outer surface of the catheter.

The proximal end of the infusion lumen is coupled in fluid communication with an outlet orifice of an infusion delivery device for controlled infusion of a therapeutic drug or diagnostic agent. This infusion delivery device can be a syringe pump, a drug infusion pump, or a similar drug delivery pump. The delivery device may also include a sensor for measuring a quantity of the medicinal agent delivered. The system also comprises an external constrictor that applies pressure to an external portion of the limb, between the patient's heart and the treatment site, to occlude the outflow of blood from the treatment site. The constrictor preferably includes a pressure cuff that is inflated with an inflation pump, or alternatively, comprises a tourniquet adapted to wrap around a limb of the patient and to apply compression to the limb. Optionally, a pressure sensor is included for measuring the pressure provided by the inflation pump, if the pressure cuff is employed.

An automated embodiment of the system comprises a controller connected to the infusion delivery device to regulate the flow of medicinal agent to the catheter and to the constrictor, to control pressurization of the constrictor. One form of the controller includes simple timers that determine time intervals for energizing the drug infusion delivery device and a pressurized fluid source that pressurizes the constrictor, while another form of the controller comprises a processor that executes machine instructions to control the repetitive constriction and drug infusion process. Preferably, the controller implements a rest/pressure timer function to determine a rest period (the period during which the pressure applied to the constrictor is released) and a pressurization period, to automatically pressurize and release the pressure applied to the constrictor at predetermined intervals. The controller also preferably automatically determines when the delivery device should be activated and deactivated. For example, the delivery device can be activated at the same time that the constrictor is activated, or after a predetermined delay following activation of the constrictor, or when a predetermined pressure has been applied by the constrictor. The controller may further automatically activate the delivery device for a predetermined dosage time period or until a predetermined dose of the medicinal agent has been delivered.

Another aspect of the invention is directed to a method for controlling delivery of a medicinal agent to a treatment site within a limb of a patient through a lumen of a catheter that has been inserted into a blood vessel of the patient and advanced to the treatment site. The method comprises the steps of activating a constrictor that applies an external pressure to stop the flow of blood within the limb in which the treatment site is disposed, and activating a delivery device that is adapted to deliver the medicinal agent to the treatment site through the catheter to keep the medicinal agent at the treatment site at least while the blood flow in the limb is stopped. Preferably, the constrictor is automatically activated for a predetermined constriction period to stop the flow of blood while the delivery device is automatically activated for a predetermined infusion period or as necessary to infuse a predetermined dose of the medicinal agent. The delivery device may be deactivated if a total quantity of the medicinal agent delivered equals a predetermined limit. After the medicinal agent is delivered, the method further includes the steps of deactivating the constrictor to allow blood flow to resume in the limb and the treatment site during a rest period. Following the rest period, the above steps are optionally repeated for a desired number of cycles.

Another aspect of the invention is directed to a machine readable medium on which are stored machine readable instructions, which when executed by a processor, cause it to perform functions generally consistent with the steps described above.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 is an illustration of a patient's leg, with a catheter inserted into a vein and a flow restricting cuff applied to the leg, in accord with the present invention;

FIG. 1A is an enlarged cross-sectional view of the catheter of FIG. 1;

FIG. 2 is a schematic diagram of a system for localized drug delivery through an artery to a treatment site in the foot of a patient, in accord with the present invention;

FIG. 3 is a block diagram of a controller for automatically providing repetitive delivery of a medicinal agent to a treatment site in a limb of the patient;

FIG. 4 is a block diagram of a processor-based controller for automatically providing repetitive delivery of a medicinal agent to a treatment site in a limb of the patient; and

FIG. 5 is a flow chart of the control logic implemented by the controller of FIG. 4, to control the repetitive delivery of medicinal agent to the treatment site.

### DESCRIPTION OF THE PREFERRED EMBODIMENT

The drawings illustrate both the design and utility of several preferred embodiments of the present invention. Similar elements of the different embodiments are referred to by the same reference numbers to simplify the description of each preferred embodiment.

As used herein, the term “medicinal agent” refers to any therapeutic agent or any diagnostic agent that might be infused to an internal treatment site within a limb of a patient's body. The term “therapeutic agent” refers to any chemical or other material that is used in the treatment of a disease or disorder. Examples, without limitation, of therapeutic agents are antibiotics, chemotherapy agents, gene therapy agents, anti-neoplastics, hormones, antivirals, radiation sources (such as cobalt, radium, radioactive sodium iodide, etc.), anticoagulants, enzymes, hepatoprotectants, vasodilators, prodrugs, and the like. Any therapeutic agent that is a fluid, or can be dissolved in a fluid, or carried in suspension by a fluid may be administered using the present invention.

As used herein, the term “diagnostic agent” refers to any chemical or other material that is used to determine the nature of a disease or disorder. Examples, without limitation, of diagnostic agents are dyes that react with metabolic products of a particular disease, and radioactive materials that bind to and thereby indicate the presence of disease-causing entities within a patient's body. As is the case with therapeutic agents, any diagnostic agent that is a fluid, or can be dissolved in a fluid, or carried in suspension by a fluid may be employed using the devices and methods herein.

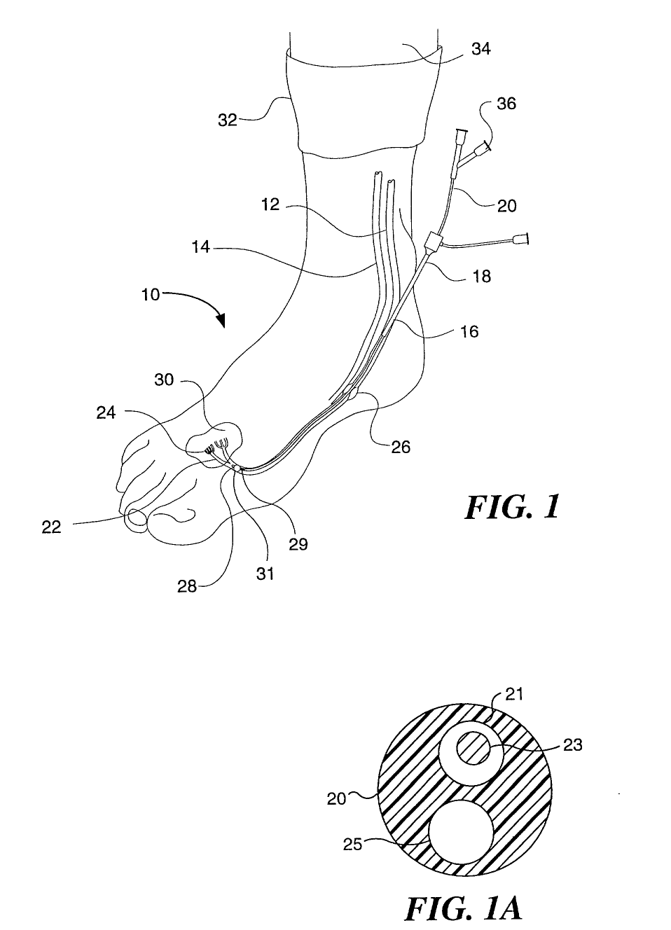

FIG. 1 illustrates a first preferred embodiment of the present invention as used to infuse a medicinal agent to a treatment site within a foot 10 of a patient. Within foot 10 are generally parallel extending veins 12 and 14 that drain blood from the lower leg and foot. Inserted into vein 12 at a puncture site 16 is an introducer sheath 18 that facilitates insertion of a catheter 20, which delivers a medicinal agent to a treatment site 30. The medicinal agent is to be delivered to the treatment site, which is disposed proximate the end of vein 12, where the vein divides into smaller venules 22, that further subdivide into capillary vessels 24. To reach this location, catheter 20 is guided through introducer sheath 18 and advanced retrograde through a venous valve 26 within vein 12 until a catheter distal tip 28 is disposed adjacent to treatment site 30. As shown in FIG. 1A, catheter 20 includes a lumen 21 through which a guide wire 23 may be inserted to assist in guiding the catheter to a desired position within a vessel. Also included is a lumen 25 for use in infusing a medicinal fluid. Catheter distal tip 28 may optionally include a radio-opaque element 29 that is readily visible in X-ray images, to assist in advancing and positioning the catheter distal tip adjacent to the treatment site. Additionally, details of the steps involved in advancing a catheter in a retrograde direction within veins containing valves are described in commonly assigned U.S. patent application, Ser. No. 09/595,853, entitled “Methods of Catheter Positioning and Drug Delivery in Veins Containing Valves,” filed on Jun. 16, 2000, the drawings and specification of which are hereby specifically incorporated herein by reference.

Once distal tip 28 is positioned in the desired location adjacent to the treatment site, blood flow within the foot is stopped by a flow restricting cuff 32 that is placed around a calf 34 on the patient's leg. The flow restricting cuff may comprise a conventional tourniquet or more preferably, comprises a elastomeric annular chamber that is secured around the limb of the patient and then pneumatically inflated—either manually with a squeeze bulb pump (not shown), or automatically with a pneumatic pump that is electrically energized. Sufficient constriction force is exerted by the flow restricting cuff (or tourniquet) to interrupt blood flow into and out of the limb on which the constricting device is fastened. The flow restricting cuff is similar to the cuff typically applied to a patient's upper arm by medical practitioners when measuring blood pressure. When blood flow within the foot (i.e., flow into and out of the foot) has been stopped by the flow restricting cuff, a medicinal agent (not shown) is administered to the treatment site through lumen 25 of catheter 20, by forcibly injecting the medicinal agent through a Luer fitting 36 that is in fluid communication with the lumen. An enlarged spherical portion 31 of the catheter is formed adjacent to distal tip 28 to seal against the interior surface of the blood vessel and block the medicinal agent from flowing back past the external surface of the catheter within the blood vessel in which the catheter is disposed. Because the blood flow within foot 10 has been stopped, and because the drug is delivered at a precise location adjacent to the treatment site, more of the drug is absorbed by the target tissue at the treatment site than if the drug were externally injected into foot 10 or into the vascular system of the patient. Also, the medicinal agent is administered only to the treatment site. Any concern about the toxicity or other adverse effect of the medicinal agent on the patient's body is minimized, since the medicinal agent has been infused at the treatment site, where its desired action is required and because it will not be conveyed by blood throughout the patient's systemic system, at least until after it has completed its intended function. A relatively small amount of the medicinal agent can be used when infused directly into the treatment site, compared to the much larger dose that would be required if the medicinal agent were simply injected into the patient's body or if administered orally. Accordingly, any toxic or other adverse effects of the medicinal agent on the patient's body are substantially avoided by the present invention as a result of the relatively low dosage of the medicinal fluid required.

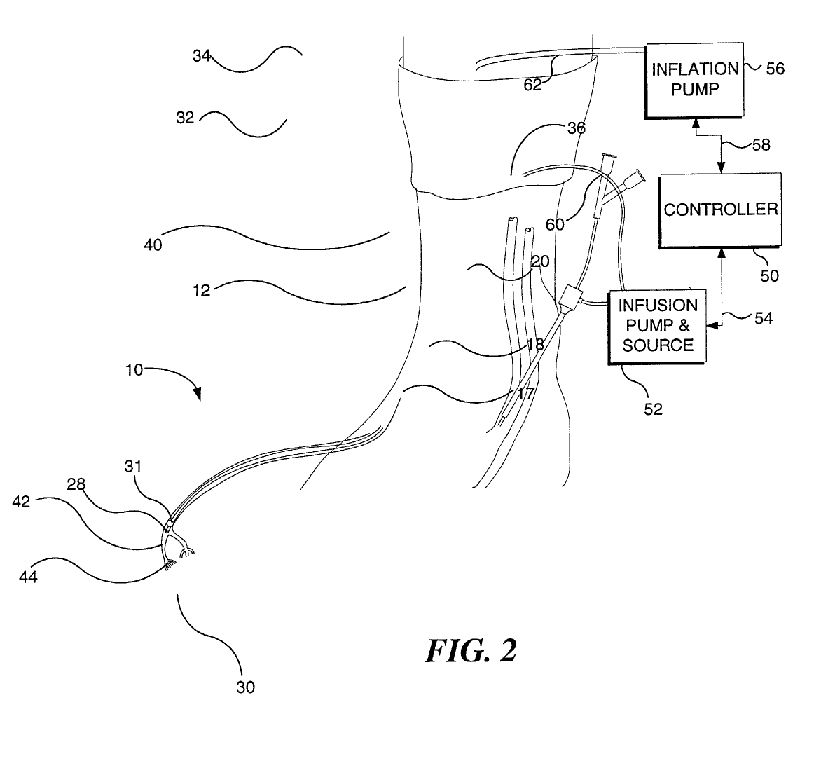

Referring to FIG. 2, a similar preferred embodiment of the present invention is shown in which catheter 20 is advanced antegrade to treatment site 30 through an artery 40 instead of vein 12. Introducer sheath 18 has been inserted into artery 40 at a puncture site 17, facilitating insertion of catheter 20. Proceeding distally down the limb of the patient, artery 40 divides into smaller arterioles 42, which still more distally, divide into capillary vessels 44. Catheter 20 has been advanced antegrade down artery 40, until catheter distal tip 28 is at treatment site 30. Flow restricting cuff 32 is again disposed around calf 34, to obstruct the flow of blood into and out of foot 10 while the medicinal fluid is being administered to the treatment site through catheter 20.

Applicable to both the venous and arterial applications of the present invention shown respectively in FIGS. 1 and 2 is a controller 50 that can automatically control blood flow into and out of the affected limb and administer a medicinal agent to the treatment site in the limb at predetermined intervals of time. While a conventional personal computer or other more general computing device (not shown) can be used for controlling and automating the repetitive infusion of the medicinal agent through catheter 20 and controlling the pressurization of cuff 32, it is likely that controller 50 will be specifically designated for this purpose. The controller may be battery powered (not shown) or powered from an internal or external alternating current line power supply (not shown), such as a conventional transformer “power brick” of the type commonly used to provide power to computer peripheral devices. Controller 50 controls an infusion pump and source 52 via signals conveyed over an infusion control line 54 and controls an inflation pump 56 via signals conveyed over an inflation control line 58.

Luer fitting 36 on catheter 20 is coupled in fluid communication with infusion pump and source 52 through an infusion line 60. The infusion pump and source includes a small reservoir, vial, or other container (not separately shown) in which the medicinal agent is stored. When the medicinal agent is administered manually, a conventional syringe can be used to force the medicinal fluid through the catheter to the treatment site. In the automated embodiment shown in FIG. 2, infusion pump 52 preferably comprises an automated syringe pump, a cassette pump, peristaltic pump, or other suitable medicinal fluid pump that is controlled in response to a signal received from controller 50 over infusion control line 54. Inflation pump 56 is connected in fluid communication with flow restricting cuff 32 via a flexible tube 62. Inflation pump 56 may comprise a standard pneumatic inflation pump of a size and volumetric rating suitable for pneumatically inflating flow restricting cuff 32 to a pressure sufficient to substantially stop blood flow into and out of a limb of a patient, in response to a signal received from controller 50 over inflation control line 58.

Once the system has been set up as shown, flow restricting cuff 32 is inflated to stop blood flow into and out of the limb for a specific (predefined) length of time. Once the appropriate inflation pressure is reached to stop blood flow in the limb, infusion of the medicinal agent (not shown) commences with the delivery of a specific bolus of the medicinal agent from infusion pump and source 52 through line 60 and catheter 20, into treatment site 30. After a predetermined infusion time period has elapsed, inflation pump 56 releases the pressure in flow restrictive cuff 32, and blood flow is restored to the leg and foot of the patient. After a specific rest period, the inflation and infusion sequence is repeated. In this manner, small doses of medicinal agent are repetitively safely infused into the treatment site.

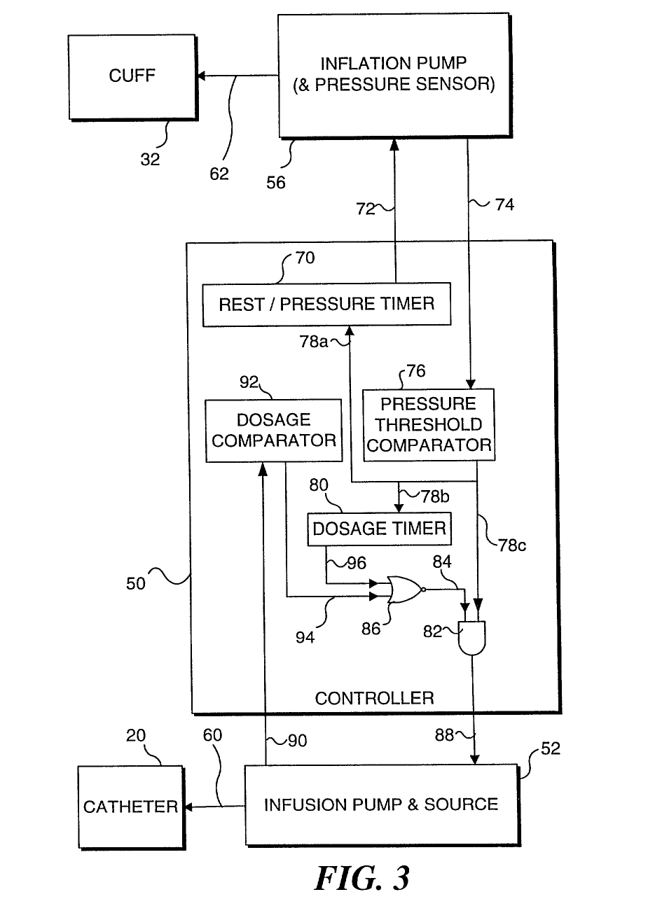

FIG. 3 is a block diagram of a preferred embodiment of controller 50 for providing such repetitive delivery of a medicinal agent. A rest/pressure timer 70 provides an inflate/deflate signal over a line 72 to inflation pump 56 to control the pneumatic pressure in tube 62 and flow restrictive cuff 32. The inflate/deflate signal can optionally be used to start a delay timer (not shown), to provide a delay after starting to pressurize flow restrictive cuff 32, before activating infusion pump 52. Alternatively, the infusion pump can be activated directly by the signal that start the pressurization of flow restrictive cuff, if the infusion pump is designed to infuse the medicinal agent sufficiently slowly that the flow of blood in the limb is substantially interrupted before any significant amount of the medicinal agent is infused into the treatment site. Preferably, however, a pressure sensor (not separately shown) in inflation pump 56 returns a pressure signal on a line 74 that is indicative of the pneumatic pressure applied to flow restrictive cuff 32. The pressure signal on line 74 is supplied to a pressure threshold comparator 76, which determines when the pressure in the flow restrictive cuff has reached a threshold level that is sufficient to stop the flow of blood in the limb of the patient. When the pressure threshold is reached, pressure threshold comparator 76 provides a full-pressure signal on a line 78a that is coupled to rest/pressure timer 70, which determines the time that inflation pump 56 is to maintain this pressure within flow restrictive cuff 32. The time interval for maintaining the pressure is preferably about ten minutes. The full-pressure signal is also provided over a line 78b to a dosage timer 80, which determines the time interval for infusing the medicinal agent into the treatment site. This time interval is thus initiated when the full-pressure signal indicates that the flow of blood in the limb has been stopped.

Pressure threshold comparator 76 also provides the full-pressure signal over a line 78c to an infusion logic gate 82 to indicate that the pressure in the flow restrictive cuff is adequate to stop the flow of blood in the limb, so that the medicinal agent can then be infused into the treatment site in the patient. In this embodiment, infusion logic gate 82 is an AND gate with two inputs and one output. Infusion logic gate 82 also receives a full-dosage signal on a line 84 from a dosage logic gate 86. In this embodiment, dosage logic gate 86 is a NOR gate providing an inverse full-dosage signal on line 84. This illustrates that until the full dosage is reached, the logic level of the full-dosage signal on line 84 remains high, enabling infusion logic gate 82 to toggle based on the full-pressure signal on line 78c. When the logic level of the full-pressure signal on line 78c is high, infusion logic gate 82 provides a high logic level infusion signal on a line 88 to infusion pump 52, enabling it to be energized. Those of ordinary skill in the art will realize that many different logic circuits and components may be combined to achieve the same result.

When infusion signal 88 is high, infusion pump 52 delivers the drug through tube 60 to catheter 20, which is routed to the treatment site. A total flow transducer (not shown) in infusion pump 52 returns a flow signal on a line 90 that is indicative of the quantity of medicinal agent delivered to the treatment site through catheter 20. A dosage threshold comparator 92 determines when the dose of medicinal agent delivered to the treatment site is equal to a predetermined level for one cycle of infusion. Dosage threshold comparator 92 can be used to control the infusion of a desired quantity of the medicinal agent during each infusion cycle, as a metering device with a variable setting to regulate the flow rate of the medicinal agent administered to the treatment site to a desired level for a predetermined time, or can be used as an emergency shut-off, to prevent an excessive quantity of the medicinal agent from being administered. When the predetermined dosage level or threshold is reached, dosage threshold comparator 92 provides a full-quantity signal on a line 94 to infusion logic gate 82. A high logic level full-quantity signal on line 94 causes the output of dosage logic gate 86 to go low, which causes the output of infusion logic gate 82 to go low, stopping infusion pump 52 from delivering any more of the medicinal agent to the treatment site.

Similarly, when dosage timer 80 determines that the time interval during which the medicinal fluid is to be administered has elapsed, the dosage timer provides a dosage time-out signal on a line 96 to infusion logic gate 82. The drug delivery time interval is preferably the same as the pressure duration, i.e., approximately ten minutes. A high logic level dosage time-out signal on line 96 also causes the output of dosage logic gate 86 to go low, which causes the output of infusion logic gate 82 to go low, stopping infusion pump 52 from delivering any more of the medicinal agent.

When an infusion period is complete, rest/pressure timer 70 provides a signal over line 72 to inflation pump 56 that causes the inflation pump to release the pressure in flow restrictive cuff 32, so that blood flow in the limb of the patient resumes. Rest/pressure timer 70 then initiates a predetermined rest period, and after the rest period, begins the pressurization and infusion cycle again. Controller 50 may also includes a cycle counter (not shown) that counts the cycles until a desired number of cycles of medicinal fluid infusion have been achieved, which causes the repetitive process of medicinal fluid infusion to be stopped.

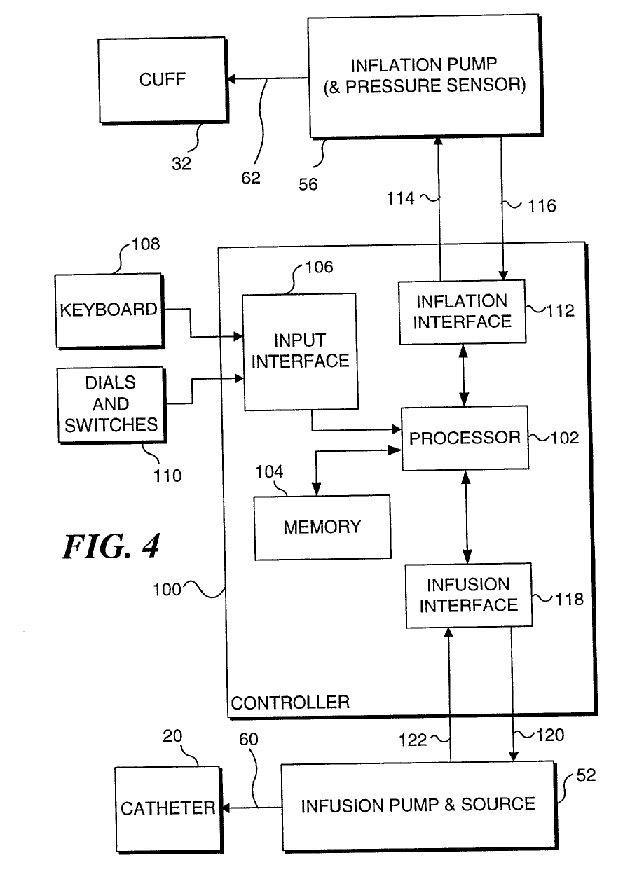

FIG. 4 illustrates a processor-based controller 100 that automates the delivery of a medicinal agent to a treatment site. Controller 100 may be a specialized device designed specifically for the purpose of controlling delivery of the medicinal agent to a treatment site, or a general computing device, such as a personal computer that is programmed to do so. Controller 100 includes a processor 102, which may be a microcontroller if controller 100 is a specialized device, or may be a typical processor of the type commonly used in a personal computer. Processor 102 is coupled to a memory 104 in which machine instructions and data are stored. Memory 104 will include volatile memory or random access memory (RAM) and non-volatile memory or read only memory (ROM). Controller 100 may also include a permanent storage (not shown), such as a hard disk, and a removable storage medium drive (not shown), such as a floppy disk drive. Also connected to processor 102 is an input interface 106, which provides communication with a keyboard 108, which may be a specialized keypad with control specific functional buttons, or a general purpose computer keyboard. Keyboard 108 is used to enter commands and parameters used to control delivery of a medicinal agent to a treatment site. Displays and switches 110 are additionally or alternatively used to enter commands and parameters to control delivery of the medicinal agent. For example, displays and switches 110 may be used to manually enter a constriction time period, a rest period, a number of cycles, a dosage of the medicinal agent per cycle, a maximum total allowable dosage, and the like.

Processor 102 also communicates with an inflation interface 112, sending activation and deactivation commands to the inflation interface, and receiving pressure data from it. Inflation interface 112 optionally includes an analog-to-digital converter (ADC) (not separately shown) for converting the analog pressure signals produced by a pressure sensor included in inflation pump 56, to corresponding digital pressure data. The processor produces a command signal 114 to control inflation pump 56. As explained above, inflation pump 56 provides pressurized air through tube 62 to cuff 32 to inflate the cuff sufficiently to constrict the flow of blood in the limb of a patient.

In addition, processor 102 communicates with an infusion interface 118, sending activation and deactivation commands, and receiving medicinal agent flow data. Infusion interface 118 sends a command signal 120 to infusion pump 52 to activate and deactivate the infusion pump. An ADC (not shown) is provided for receiving an analog flow signal 122 produced by a flow sensor (not shown) in the infusion pump and converting the analog signal to digital data. As explained above, infusion pump 52 delivers the medicinal agent through tube 60 and through catheter 20 to the treatment site in the limb of the patient. Infusion pump 52 may also include a reservoir, vial, or other source (not separately shown) for the medicinal agent, or may comprise a motorized syringe that simply delivers the medicinal agent contained within the syringe by advancing a plunger (not shown).

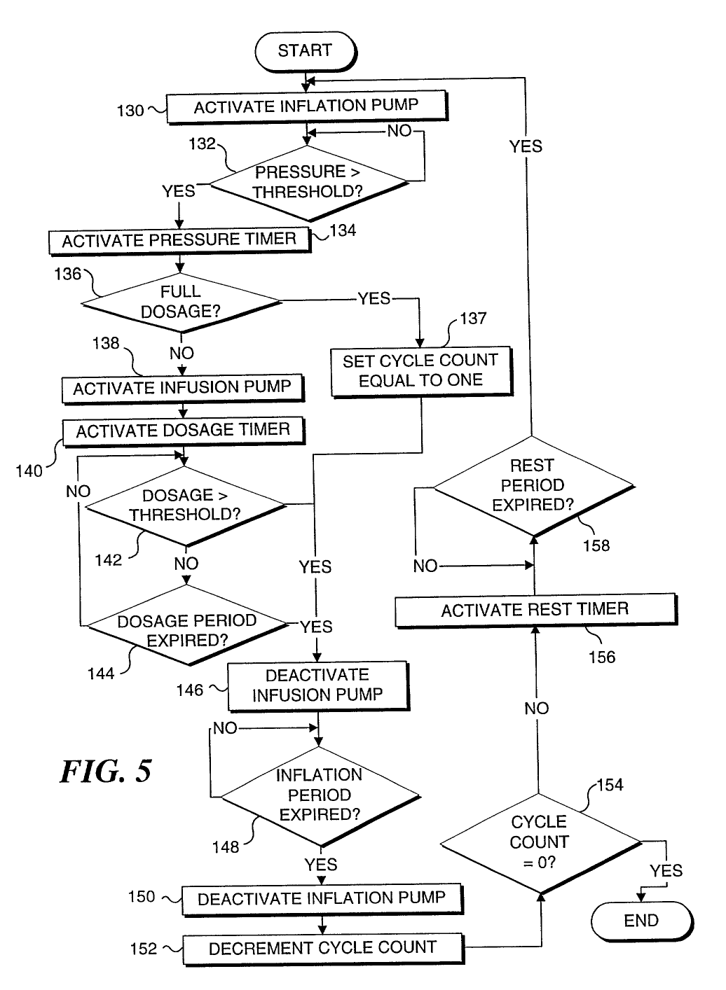

FIG. 5 illustrates the control logic that is implemented in software or in hardwired logic to control repetitive cycles of infusion of a medicinal agent to a treatment site in a limb of a patient. At a block 130, inflation pump 56 is activated to pressurize flow restrictive cuff 32 to stop the flow of blood in the affected limb. At a decision block 132, the pneumatic pressure in the flow restrictive cuff is compared with a predetermined pressure threshold value to determine if the pressure in the flow restrictive cuff is sufficient to stop blood flow in the limb. This decision repeats until the detected pressure value in the flow restrictive cuff is greater than the pressure threshold value. A pressure timer is then activated at a block 134 to begin a predetermined inflation period during which the flow of blood in the limb is stopped.

When the detected pressure value is greater than the pressure threshold value, a decision block 136 determines if a predetermined total dosage of the medicinal agent has been administered. The amount of medicinal agent administered is determined by a flow transducer in the infusion pump or other sensor and compared with a predetermined dosage value that can be set by a medical practitioner as a desired dosage or as a maximum allowable dosage. If a full dosage of the drug has already been administered so that no further drug infusion is required by the infusion pump, then the cycle count is set to one in a block 137 and the infusion pump is deactivated at a block 146. The logic can be modified so that a full dosage result will also deactivate the inflation pump, as indicated in a block 150, causing blood flow in the limb to be immediately enabled. However, the logic shown maintains the pressure in the flow restrictive cuff for the entire inflation period so that any manually administered drug may be perfused into the tissue of the treatment site without blood flow carrying away the drug.

If a full dosage is not detected, the infusion pump is activated in a block 138, and a dosage timer is activated in a block 140. A decision block 142 determines whether the quantity of the medicinal agent delivered during a current cycle has reached a desired dosage threshold. If so, then the infusion pump is deactivated in block 146. If the dosage threshold for the current cycle has not been reached, a decision block 144 determines whether a dosage period has expired, as established by the dosage timer. If the dosage period has not yet expired, the drug delivered is checked again at decision block 142. When the dosage period has expired, the infusion pump is deactivated at block 146.

A decision block 148 then determines whether a current inflation period has expired, as determined by the pressure timer. Until the inflation period has elapsed, the logic loops, providing time for the delivered drug to perfuse the tissue of the treatment site while the blood flow is stopped. Once the inflation period has expired, the inflation pump is deactivated at step 150.

Block 150 concludes a cycle of medicinal agent infusion, so a cycle counter is decremented in a block 152. A decision block 154 then determines whether all of a predetermined number of cycles of infusion of the medicinal agent have been completed. If all of the infusion cycles have been completed, the process ends. If additional infusion cycles remain, the rest timer is activated in a block 156. A decision block 158 then determines whether the rest period has expired, as determined by the rest timer. Until the rest period has elapsed, the logic loops, providing time for blood to resume flowing in the limb and treatment site, re-oxygenating tissue in the portion of the limb where blood flow was prevented. Once the rest period has expired, the next infusion cycle begins by reactivating the inflation pump at step 130.

Although the present invention has been described in connection with the preferred form of practicing it and modifications thereto, those of ordinary skill in the art will understand that many other modifications can be made to the present invention within the scope of the claims that follow. Accordingly, it is not intended that the scope of the invention in any way be limited by the above description, but instead be determined entirely by reference to the claims that follow.

The invention in which an exclusive right is claimed is defined by the following:

1. A method for delivering a medicinal agent to a treatment site within a limb of a patient, comprising the steps of:
(a) inserting a catheter into a blood vessel of the patient and advancing the catheter through the blood vessel until a distal tip of said catheter is disposed adjacent to the treatment site;
(b) stopping blood flow within the limb by applying an external pressure to the limb; and
(c) delivering the medicinal agent to the treatment site through the catheter so that the medicinal agent infuses the treatment site and remains at the treatment site at least while the blood flow in the limb is stopped.

2. The method of claim 1, further comprising the steps of:
(a) retaining the medicinal agent at the treatment site for a predetermined perfusion time to allow perfusion of the medicinal agent into tissue proximate the treatment site; and
(b) removing said external pressure to reestablish blood flow in the limb, after the predetermined perfusion time has elapsed.

3. The method of claim 2, wherein the perfusion time is less than about ten minutes.

4. The method of claim 2, further comprising the steps of:
(a) enabling blood flow to resume after the perfusion time, for a predetermined rest period, to re-oxygenate tissue within the limb;
(b) again stopping the blood flow within the limb by re-applying the external pressure; and
(c) again delivering the medicinal agent to the treatment site through the catheter.

5. The method of claim 1, wherein the step of stopping blood flow within the limb comprises the step of tightening a tourniquet around the limb of the patient at a location proximal to the treatment site.

6. The method of claim 1, wherein the step of stopping blood flow within the limb comprises the step of inflating a pressure cuff around the limb of the patient at a location proximal to the treatment site.

7. The method of claim 1, wherein the step of inserting the catheter comprises the step of inserting the catheter into a vein of the patient and advancing the catheter in a retrograde direction within the vein until the distal end of the catheter is disposed adjacent to the treatment site.

8. A system for delivering and retaining a medicinal agent at a treatment site within a patient, comprising:
(a) an infusion catheter having a lumen that extends to a distal end of the infusion catheter from a port disposed adjacent to a proximate end of the catheter, said infusion catheter being adapted for insertion into a blood vessel of a patient and adapted to be advanced to a treatment site; and
(b) an external constrictor that is adapted to exert sufficient constrictive pressure on a limb of a patient to stop blood flow within the limb while a medicinal agent is infused into a treatment site to which the distal end of the catheter has been advanced through a blood vessel of a patient, so that the medicinal agent remains at a treatment site at least while a blood flow in a limb is stopped.

9. The system of claim 8, further comprising a delivery device connected in fluid communication with a proximal end of the lumen of the infusion catheter, said delivery device being employed to infuse the medicinal agent into the treatment site through the lumen.

10. The system of claim 8, further comprising a controller connected to control operation of said delivery device and said constrictor, wherein said controller automatically causes said constrictor to stop a blood flow within a limb of a patient, and wherein said controller automatically activates said delivery device, causing the medicinal agent to be infused at the treatment site.

11. The system of claim 10, wherein said controller repetitively activates and deactivates said constrictor and said delivery device so that the medicinal agent is infused into the treatment site during successive cycles.

12. The system of claim 9, wherein said delivery device comprises an infusion pump that is activated by said controller.

13. The system of claim 12, wherein said delivery device further comprises a flow sensor that produces a signal indicative of a flow of the medicinal fluid into the lumen, for use in determining a quantity of the medicinal agent delivered to the treatment site, said signal being supplied to the controller for use in controlling the delivery device.

14. The system of claim 8, wherein said constrictor comprises a fluid actuated cuff adapted to wrap around a limb of a patient; further comprising an inflation pump operatively connected with said controller for receiving an activation signal from said controller, and wherein said inflation pump is operatively connected to said cuff to provide fluid for inflating said cuff.

15. The system of claim 14, wherein said constrictor further comprises a pressure sensor for detecting a pressure applied to the fluid actuated cuff, which is indicative of a pressure applied to a limb of a patient.

16. The system of claim 11, wherein said controller comprises a timer that determines time intervals, including at least one of:
(a) a pressure time interval during which the controller causes a blood flow to be stopped in a limb of a patient;
(b) a rest time interval between successive cycles; and
(c) a dosage time interval during which the medicinal fluid is infused into the treatment site.

17. The system of claim 16, wherein said controller determines a total dosage of the medicinal agent that is delivered to the treatment site.

18. The system of claim 16, wherein said controller causes the delivery device to deliver a predetermined dosage of the medicinal agent.

19. The system of claim 8, wherein said infusion catheter includes a radio-opaque element disposed adjacent to its distal end to assist in positioning the distal end adjacent to the treatment site.

20. The system of claim 8, wherein said infusion catheter includes an enlarged distal portion adjacent to the distal end of the infusion catheter for sealing against an inner wall of a blood vessel to prevent the medicinal agent from flowing back along the infusion catheter and away from the treatment site.

21. The system of claim 8, wherein said infusion catheter includes a second lumen adapted to receive a guide wire to assist in positioning the distal end of the infusion catheter.

22. The system of claim 8, further comprising an introducer sheath, adapted for insertion into a blood vessel of a patient, to provide a reusable access site for insertion of the infusion catheter.

23. A method for controlling delivery of a medicinal agent to a treatment site within a limb of a patient through a catheter that has been inserted into a blood vessel of the patient and advanced to the treatment site, comprising the steps of:
(a) automatically activating a constrictor, causing the constrictor to apply an external pressure to the limb of the patient to stop the flow of blood within the limb; and
(b) automatically activating a delivery device to deliver the medicinal agent to the treatment site through the catheter, said medicinal agent remaining at the treatment site at least while the flow of blood within the limb is stopped.

24. The method of claim 23, wherein the step of causing the constrictor to apply the external pressure comprises the step of releasing the external pressure after a predetermined constriction period of time has elapsed.

25. The method of claim 23, wherein the step of automatically activating the delivery device comprises the step of infusing the medicinal agent for a predetermined infusion period of time.

26. The method of claim 23, further comprising the step of delivering a predetermined dosage of the medicinal agent to the treatment site.

27. The method of claim 23, further comprising the steps of:
(a) detecting a quantity of the medicinal agent delivered to the treatment site; and
(b) determining when to deactivate said delivery device as a function of the quantity of the medicinal agent that has been delivered to the treatment site.

28. The method of claim 23, further comprising the step of deactivating said delivery device once a total quantity of the medicinal agent delivered to the treatment site during one or more cycles of delivery of the medical agent equals a predetermined threshold limit.

29. The method of claim 23, further comprising the steps of:
(a) repeating steps (a) and (b) in a plurality of successive cycles; and
(b) allowing blood flow to resume in the limb for a predetermined rest period between successive cycles.

30. A machine readable medium on which are stored machine readable instructions for performing the steps of claim 23.
